(19) United States
(12) Patent Application Publication
(10) Pub. No.: US 2021/0007327 A1
(43) Pub. Date: Jan. 14, 2021

(54) Smart Delivery Treatment Apparatus For Remote Treatment Of An Animal

(71) Applicant: SmartVet Pty Ltd, Kenmore East, Queensland (AU)

(72) Inventor: Grant Andrew WEYER, Noosa Heads, Queensland (AU)

(73) Assignee: SmartVet Pty Ltd, Kenmore East, Queensland (AU)

(21) Appl. No.: 16/979,980

(22) PCT Filed: Mar. 15, 2019
(86) PCT No.: PCT/AU2019/000038

§ 371 Date: Sep. 11, 2020

Related U.S. Application Data
(60) Provisional application No. 62/643,312, filed on Mar. 15, 2018.

(51) Int. Cl. A01K 13/00; A61D 7/00; A01K 11/00; B64C 39/02; G05D 1/00; G05D 1/10; G05D 1/02; G16H 40/63; G16H 20/17; F42B 12/36; F41B 11/85

(52) U.S. Cl. CPC A01K 13/003; A61D 7/00; A01K 11/006; B64C 39/02; G05D 1/0094; G05D 1/101; B64C 2201/12; G05D 1/021; G16H 40/63; G16H 20/17; F42B 12/362; F41B 11/85; G05D 1/0011

(57) ABSTRACT

The present disclosure is directed to an apparatus for remotely treating an animal with an agent comprising: a launcher adapted to hold an agent and propel the agent in a treatment dose over a distance to an animal in response to a trigger command; an electronic reader module comprising: a communication interface arranged to receive an identification signal from a sensor associated with an animal; a trigger interface arranged to detect a trigger command to the launcher; and a means to record in a memory the treatment of the animal identified from electronic sensor and treated by the launcher.

### TECHNICAL FIELD

The technology relates to a remote delivery treatment apparatus for treatment of animals such as livestock.

### BACKGROUND

Efficient delivery and management of treatment to animals such as livestock can be challenging, in particular, on farms where animals are free to move across vast portions of land, such as many beef cattle farms. It is also challenging for farmers to efficiently identify specific animals that require treatment and deliver the correct medical treatment.

Prior art methods require the farmer to plan which animals to treat and, for each animal, make a note of what treatment is to be administered. Furthermore, the animals have to be located and often gathered and moved to a pen, or similar handling facility, where they are identified, often visually by reading their ID number or brand. The animals are then treated with the required medicinal compounds. After the treatment has been administered, farmers need to create a record of the type and time of treatment provided to the specific animal.

Veterinary compounds, pesticides and vaccines can be administered to animals in different ways. For example, animals can be gathered and confined in special handling facilities where treatment can be administered by injection, pour-on or spray. In some instances, where cattle are too sick to be moved or where safe handling facilities are not available, a specially designed dart may be used to administer an injectable medicine or vaccine. Treatment can be administered more efficiently, and with less stress for the animal, by using systems that allow for remote delivery of a variety of types of medicaments. For example, U.S. Pat. Nos. 8,425,932; 8,802,135; 9,238,001; US 2013/0303988 and US 2015/0237849, the disclosure of which is incorporated herein by reference, disclose a launching system and a method for remotely treating an animal using dosage projectiles having frangible shells.

All of the methods and systems proposed in the prior art require identification of the animal by the farmer and careful selection and recording of the treatment administered. These steps cause additional workload for the farmer while out in the field and, therefore, reduce efficiency. The prior art methods are also vulnerable to human errors made by the farmer such as recording a specific treatment against an incorrectly identified animal.

The present inventors have developed a system that facilitates the identification of an animal, provision of the appropriate treatment and recording of the treatment.

### SUMMARY

Embodiments of the present invention are directed to a treating apparatus for remotely treating an animal and a method for treating the animal using the treating apparatus that allow for identification of animals that need treatment and digital management of treatment records.

According to a first aspect of the invention there is provided an apparatus for remotely treating an animal with an agent, the apparatus comprising:

a launcher adapted to hold an agent and propel the agent in a treatment dose over a distance to an animal in response to a trigger command;

an electronic reader module comprising:
a communication interface arranged to receive an identification signal from an electronic sensor associated with an animal;

a trigger interface arranged to detect a trigger command to the launcher; and
a means to record in a memory the treatment of the animal identified from electronic sensor and treated by the launcher.

The animal may be a domestic animal or wildlife or game animal. The animal may be ovine, bovine, equine, leporine, porcine, feline, canine, caprine or avian. In an embodiment, the animal is bovine.

The apparatus may be handheld, anchored or positioned in stationary location, or associated with a mobile platform such as a vehicle, drone or cable and pulley system used to orientate it for launching.

In an embodiment the agent may be a predetermined quantity of a liquid agent. In this instance, the launcher may comprise: a chamber adapted to receive the agent; a launcher barrel or nozzle for facilitating launching of a treatment dose of the agent; and a propulsion assembly for propelling the treatment dose through the barrel or nozzle to an animal in response to the trigger command.

In an embodiment, the dose of the agent may be contained in a dosage projectile, a liquid, a spray, a dart, a gel or a bolus of powder or gel.

The dosage projectile may be a dosage dart, skin piercing dosage projectiles, non-skin piercing dosage projectile.

In an embodiment, the dosage projectile is adapted to deliver the agent to the animal substantially without piercing the skin of the animal.

In use, when an identified animal is treated with the dose of the agent launched from the launcher, the treatment is recorded and a record of treatment is stored in the memory.

The trigger interface can trigger launching of the dose by receiving an electronic command from the electronic reader, a remote electronic command or a mechanical command from a physical trigger located on the launcher.

In an embodiment, when the dose of the agent is launched by the launcher, a signal is sent from the launcher to the reader to initiate recording of the record of treatment of the animal in the memory.

In an embodiment, the identification signal received by the launcher encodes an identification code of the animal. The signal may also comprise data associated with the animal, such as identification data, treatment history, real time physiological data, health-related historical data.

The means to record the treatment can be a local memory, such as solid-state memory, hosted on the reader module, records of treatment may be stored in the local memory. Alternatively, the means to record the treatment may comprise a transmitter to transmit treatment data for recording and records of treatment may be stored in a memory embedded into the electronic sensor and the communication interface is arranged to transmit the record of treatment to the electronic sensor for storage in the memory.

In some embodiments, the electronic reader module is integrated with the launcher. In alternative embodiments, the electronic reader module is removably mounted to the launcher. The electronic reader module may be mounted onto the launcher using a mounting bracket or any other fastening mechanism.

Furthermore, in some instances, the electronic reader module may comprise two devices, a first device may be integrated with the launcher and a second device may be removably mounted onto the launcher.

In some embodiments, the communication interface is further arranged to exchange data with a personal communication device, such as a smartphone, a smartwatch or a pair of smart glasses and the record of treatment is transmitted to the personal communication device for storage.

In other embodiments, the electronic reader module comprises a personal communication device, such as a smartphone or a smartwatch, which is removably mounted to the launcher by means of a mounting bracket or a fastener. The personal communication device may interact with a detector located on the launcher. The communication interface may be hosted in the personal communication device, whilst the detector may be arranged to initiate communication between the electronic sensor on the animal and the communication interface. For example, the detector may comprise a proximity sensor which, when in proximity of the animal, initiates communication between the communication interface and the electronic sensor.

Alternatively, the electronic sensor can be activated when the detector is located within a predetermined distance and starts emitting a signal encoding identification information for the animal. The detector can read and decode the signal to identify which animal is being treated. The signal may comprise a light signal, such as a flashing light pattern, emitted by a light source that can be read by a visual system on-board of the detector.

In some embodiments, the detector is arranged to upload the record of treatment for the animal to a remote server. The remote server may be part of a health monitoring platform. The detector may be further arranged to receive data from the health monitoring platform. The data may comprise treatment history, real time physiological data, health-related historical data.

Alternatively, the detector may comprise a light emitting source, such as a laser source, that is detectable by the electronic sensor on the animal and triggers the initiation of communication between the communication interface and the electronic sensor. Once communication commences, the communication interface receives identification data from the electronic sensor and the data is prompted to the user of the apparatus.

In some embodiments, the apparatus further comprises a user interface arranged to enable user selection of a specific agent from a list of available projectile treatments that can be loaded into the launcher.

In some embodiments, upon identification of an animal, the user interface is arranged to prompt the user with a history of treatment for the animal.

The user interface may comprise a user display, such as touch screen display, to show the history of treatment for the identified animal and provide a selection menu of agents that can be loaded into the launcher.

In some embodiments, the user interface is provided on a display of the user communication device and data related to treatment of the animal are exchanged between the communication interface of the treating apparatus and the user communication device.

In a second aspect there is provided a method for remotely treating an animal, the method comprising:

providing an apparatus according to the first aspect;

identifying an animal by communication between the electronic sensor on the animal and the electronic reader module;

treating the animal by an agent dose sent from the launcher to the animal; and

recording the treatment in a memory.

In embodiments, the step of recording treatment comprises the step of transmitting the record of treatment to a remote server. In other embodiments, the step of recording treatment comprises the step of transmitting the record of treatment to the electronic sensor and storing the record of treatment in a memory of the electronic sensor.

In embodiments, the method further comprises the step of selecting a dosage projectile from a list of available projectiles available for the launcher. The step of selecting the projectile may be based on treatment history data.

In embodiments, the method further comprises the step of entering, via the user interface, a range of serial numbers of electronic sensors, associated with animals that require treatment.

In some embodiments, the method further comprises the step of confirming delivery of treatment via the user interface.

The launcher may be equipped with doses of agents, such as dosage projectiles, for different treatments. For example, the time of the year or weather conditions may be such that one or more parasites are likely to cause health problems in animals. In this case the farmer may load the launcher with agents for treatment or prevention of each specific health condition. When approaching a specific animal, the animal is identified, and data is displayed to the farmer that shows whether the animal has already been treated with one or more of the available treatments, or the animal requires treatment. The farmer can then select and administer, remotely, the treatment without need for manually identifying the animal and recording that the treatment has been administered. The data is automatically stored in the memory of the treating apparatus, in the electronic sensor of the animal or in the farmer's smartphone.

The treating apparatus and method can assist in providing numerous advantages to improve the efficiency of treatment of livestock in farms where the animals are not always located in a confined environment. The treating apparatus allows for screening of livestock and identification of required treatment. Farmers will not have to invest time in compiling lists of animals due for treatment and trying to identify each animal by visually reading an ear tag. Instead, farmers can move around the farm approaching different animals and relying on the data communication between the treating apparatus and the electronic sensor located on the animal to ascertain whether treatment is required and what type of treatment needs to be administered.

Advantages of embodiments of the system disclosed herein allow saving of precious time and resources in managing livestock on a farm. Quick identification and treatment reduces stress for the user and the animals. In addition, the capability to treat animals remotely at any time allows implementing precision medicine principles. Preventing double dosing and tracking each dose carefully enables pre-slaughter intervals to be observed. Furthermore, administered drugs can be audited and observed. Importantly, the automated identification of animals helps to avoid mistaken identity and therefore wrong treatment being administered.

In alternative embodiments, the treating apparatus may be installed on a vehicle that is capable of moving across the farm's terrain and approaching animals to deliver treatment. The vehicle may be human-controlled or autonomous. Furthermore, the vehicle may be a ground-based vehicle, an airborne based vehicle, such as a drone or suspended above the farm using a system of pulleys and cables.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all these matters form part of the prior art base or were common general knowledge in the field relevant to the present invention as it existed before the priority date of each claim of this specification.

In order that the present invention may be more clearly understood, preferred embodiments will be described with reference to the following drawings and examples.

### BRIEF DESCRIPTION OF THE DRAWINGS

Features and advantages of the present invention will become apparent from the following description of embodiments thereof, by way of example only, with reference to the accompanying drawings, in which:

FIG. 1 is a schematic diagram of a treating apparatus;

FIG. 2 is a schematic representation of a treating apparatus and an animal wearing an electronic sensor;

FIG. 3 is a schematic representation of a treating apparatus and an animal wearing an electronic sensor where the treating apparatus communicates with a smartphone;

FIG. 4 is a schematic representation of a plurality of animals in a farm and two treating apparatus, one mounted on a ground-based vehicle and the other mounted on a drone;

FIG. 5 shows a series of display screens of an application running on a smartphone that communicates with the treating apparatus;

FIG. 6 shows two screenshots of the user interface when targeting a specific animal in a farm;

FIG. 7 is a flow diagram presenting a list of steps to perform a method of remotely treating an animal and recording treatment of the animal; and

FIG. 8, is a schematic representation of a smart farm using an apparatus in accordance with embodiments.

### DESCRIPTION OF EMBODIMENTS

Embodiments of the present invention relate to a treating apparatus for remotely treating an animal and a method for treating an animal using the launcher device that allow automatic identification of animals that need treatment and digital management of treatment records.

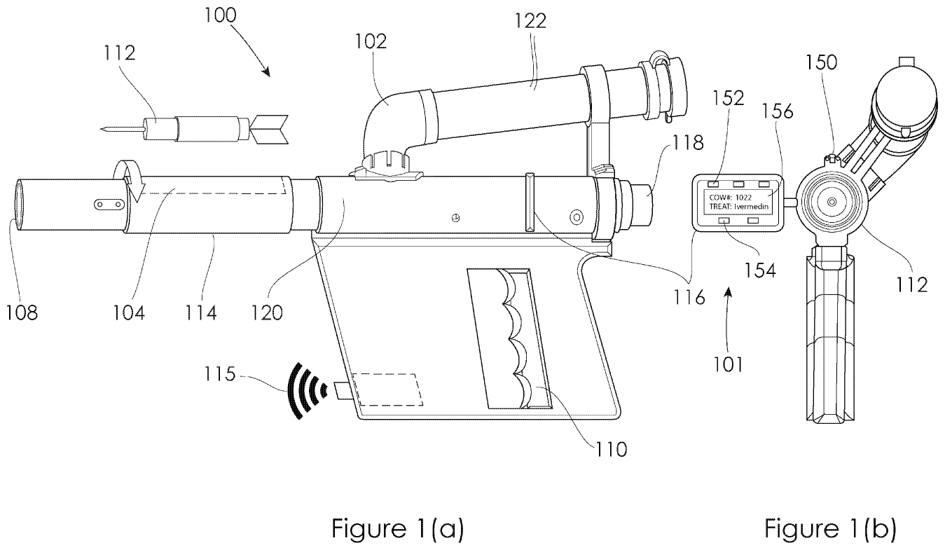

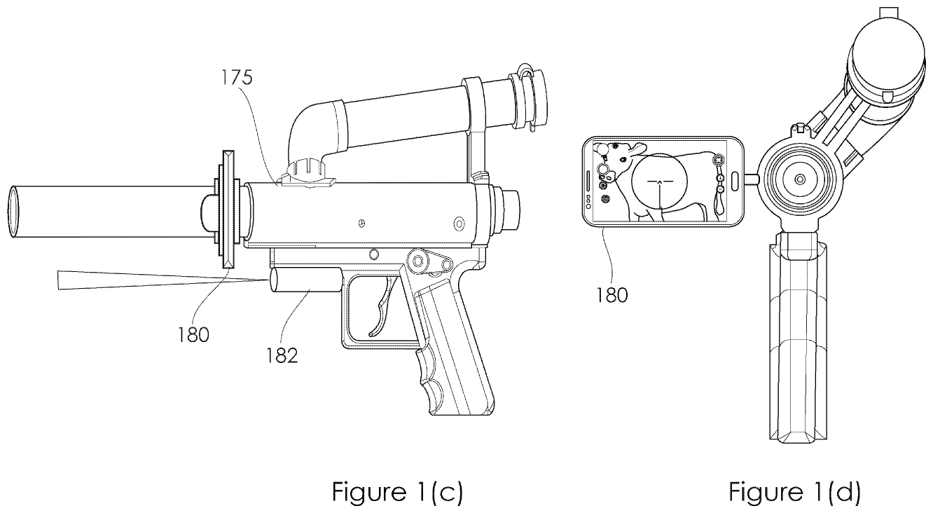

Referring now to FIG. 1(a), there is shown a schematic diagram of a treating apparatus comprising a launcher 100 and an electronic reader module 101 in accordance with an embodiment. The launcher 100 has a chamber 104 adapted to receive treating agent doses, such as dosage projectiles. Further, the launcher 100 comprises a barrel 114 for facilitating launching of the dosage projectile and a propulsion assembly for propelling the dosage projectile. The propulsion assembly for the launcher 100 of FIG. 1 is provided in the form of a gas tank attached to inlet valve 118. The gas tank may be connected to the inlet valve 118 by means of a gas line and connectors capable of carrying a relatively high-pressure gas, typically in the range of 750 psi.

Upon pressing the trigger 110, a dosage projectile is launched from the barrel 114 and out of muzzle 108. Propulsion of the dosage projectile is provided by compressed CO₂ released from a compressed air or gas tank 118. Tank 118 can be filled with nitrogen, CO₂ and/or compressed air. The trigger command can be also received from the on-board electronic reader, can be a remote electronic command or a mechanical command from the physical trigger 110.

A valved tube can be located in the body 120 which extends past a spring, bolt and hammer to the air inlet valve (not shown). A tank of compressed air is connected to the gas inlet valve 118 to provide the energy needed to launch the dosage projectile. When an actuator is cocked, a dosage projectile is advanced into the breech from a suitable loader such as hopper or loading tube 122. The spring is released when the trigger is pulled allowing a burst of air from the tank to pass through the inlet valve and launch the dosage projectile through the barrel to the animal (not shown).

The compressed air tank 118 can be filled by being attached to a larger tank that supplies the air or gas. Examples of suitable propulsion systems and applicators used in the paintball and air gun industries and suitable for the present invention can be found in U.S. Pat. No. 6,990,971, US 2010/0051008 and U.S. Pat. No. 5,823,173. It will be appreciated that spring loaded air compression arrangements can also be used for the propulsion system.

FIG. 1 shows an example of launcher that uses projectiles containing an agent to deliver treatment. Other types of launchers can be used to deliver treatment. For example, the agent can be provided as a measured fluid dose, such as a liquid or a gel, and the launcher comprise a nozzle for propelling or spraying the fluid agent towards the animal in response to a trigger command.

An electronic reader module 101 is integrated within the body of the launcher. The electronic reader module comprises a communication interface arranged to receive identification data from an electronic sensor associated with an animal. In other words, reader assembly 101 can ‘read’ the identity of the animal by communicating with the electronic sensor. In some instances, the communication interface is also arranged to transmit data to the electronic sensor associated with the animal, or to a personal communication device of the user of the launcher.

Once the treatment has been administered to the animal, wherein, when an identified animal is treated using the dosage projectile, the treatment is recorded and a record of treatment is stored in a memory.

The record of treatment is stored in the memory of the reader 101. In alternative embodiments, the record of treatment can be stored in a memory embedded into the electronic sensor on the animal after the communication interface transmits the record of treatment to the electronic sensor. In other embodiments, the communication interface is capable of exchanging data with a personal communication device, such as a smartphone. In this case, the record of treatment can be transmitted to the personal communication device for storage in the local memory of the device.

FIG. 1(b) shows a magnified rear view 150 of the treating apparatus and respective electronic reader module 101. Electronic reader module 101 comprises a user interface 152 for interaction with a user of the launcher. The user interface 152 comprises a digital display 156 to show information related to a specific animal, such as animal ID number and treatment required, if any. The display 156 may also show history data of treatment for the animal stored in the memory of the electronic reader module 101.

Furthermore, user interface 152 comprises a set of buttons 154 to navigate the functions of the launcher and allow selection of a specific projectile treatment from a list of available projectile treatments.

Display 156 and buttons 154 may be also arranged in a touch screen operable by the user of the launcher.

The electronic reader module 101 also comprises a detector module arranged to initiate communication between the electronic sensor on the animal and the communication interface. In the embodiment described, the detector has a proximity sensor 115 which, when in proximity of the animal, initiates communication between the communication interface and the electronic sensor. Once communication commences, the communication interface receives identification data from the electronic sensor and the data is prompted to the user of the apparatus.

The electronic reader 101 identifies the animal by the identification data and prompts a result of the identification to the user.

Referring now to FIG. 1(c) and FIG. 1(d) there is shown another embodiment of treatment apparatus. In this embodiment, the electronic reader module comprises a personal communication device, a detector provided as a smartphone 180 and light or sound source 182 that is detectable by the electronic sensor on the animal and triggers the initiation of communication between the communication interface, which is hosted in smartphone 180 and the electronic sensor. Smartphone 180 is removably mounted to the launcher 175 by means of a mounting bracket. Once communication commences, the communication interface receives identification data from the electronic sensor and the data is prompted to the user of the apparatus.

The communication capabilities of smartphone 180 can be used to communicate with the electronic sensor on the animal. For example, communication may occur using a Wi-Fi protocol or a long-range Bluetooth protocol.

In another example the electronic sensor in or on the animal may integrate, or be associated with, a light emitting source capable of producing a binary or other flashing light pattern as a unique identifier of the animal, the smartphone camera or visual system associated with the smartphone may recognize the binary flashing pattern to identify the animal.

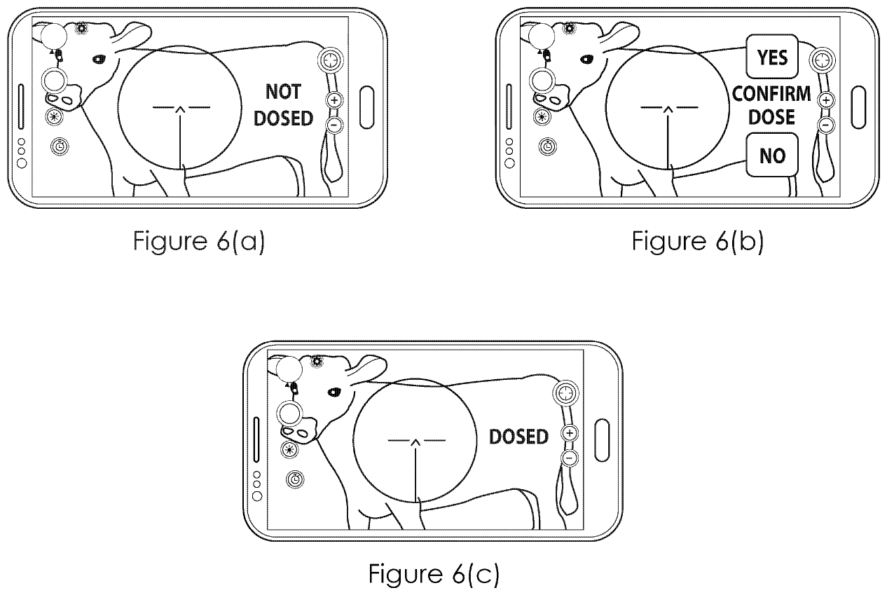

After the animal has been identified, the user interface on the screen or the smartphone 180 provides animal data for the farmer. Importantly, the user interface confirms whether the animal requires treatment or not. An example of user interface provided by a software application running on a smartphone will be discussed with reference to FIG. 6. In the instance where the animal requires treatment, the farmer can administer treatment by using the launcher 175. When the treatment is administered, a signal is captured by smartphone 180 to trigger recording of treatment for the specific animal. The signal can be a mechanical signal, such as a vibration created by the launcher 175 while launching the projectile. Alternatively, the signal can be an audio signal, such as a noise generated by the launcher 175 while launching the projectile. In other embodiments, the smartphone 180 can be electrically or wirelessly connected to the launcher 175 and receive an electrical signal after launching occurs. The mounting bracket can be configured with an electrical conductor and an electrical plug to provide the smartphone 180 electrical connection.

Treating apparatus 100 is particularly suitable for remotely treating an animal. The treating apparatus is suitable for use with a wildlife or game animal such as deer or other antelope species or buffalo, commercial or production livestock such as cattle and horses or larger bodied feral animals such as horses, buffalo wild dogs, pigs, and the like.

The animal can be treated for a condition such as disease, parasite infestation or condition, dietary deficiency, or fertility. In particular, the condition is insect or parasite infection or infestation. The animal can also be treated as a preventative measure, such as vaccination against a particular disease.

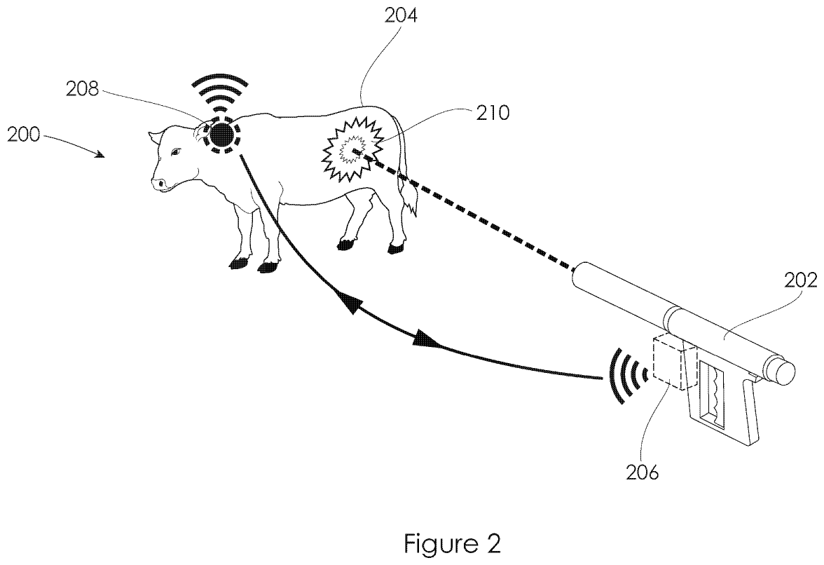

Referring now to FIG. 2, there is shown a schematic representation 200 of a treating apparatus 202 and an animal 204 wearing an electronic sensor 208. The electronic receiver module 206 on the treating apparatus is also shown. The receiver module 206 is integrated within the body of the launcher 202. In some instances, the electronic receiver module 206 can be installed on an pre-existing launcher to, for example, ‘retrofit’ launchers already on the market. This can be accomplished by, for example, using a mounting bracket and a smartphone as described above with reference to FIG. 1(c) and FIG. 1(d).

Schematic 200 shows the wireless communication between the electronic receiver 206 and the electronic sensor 208 installed on an ear of the animal 202. It will be appreciated that the electronic sensor 208 may be provided in the form of a tag, collar, subcutaneous skin implant or intra ruminal bolus among others. The communication may be initiated when proximity of the animal 204 is detected by the launcher 202. The receiver 206 and the electronic sensor 208 may communicate using a short-range communication technology, such as Radio Frequency Identification systems, short-range wireless technologies, device interconnection systems such as Bluetooth, infra-red systems or Wi-Fi or a light signal, such as a flashing light encoding animal's identification data.

Once the communication is initiated, data is transmitted from the electronic sensor 208 to the electronic receiver module 206. The user interface provides information, based on the received data, on the identity of the animal being approached. The information includes the current treatment status of the animal and whether the animal needs to be treated with a treatment projectile. The electronic sensor, in some embodiments, also provides a visual, cue, for example an LED light on the electronic sensor to visually communicate to a farmer that the animal needs treatment. In alternative embodiments, the visual cue is provided on the user interface, for example, on the display of the launcher or on the display of the smartphone, as will be discussed with reference to FIG. 6.

In case treatment is required, the farmer can select a specific treatment agent 210, using the user interface, and then load and launch the projectile 210 towards the animal for treatment.

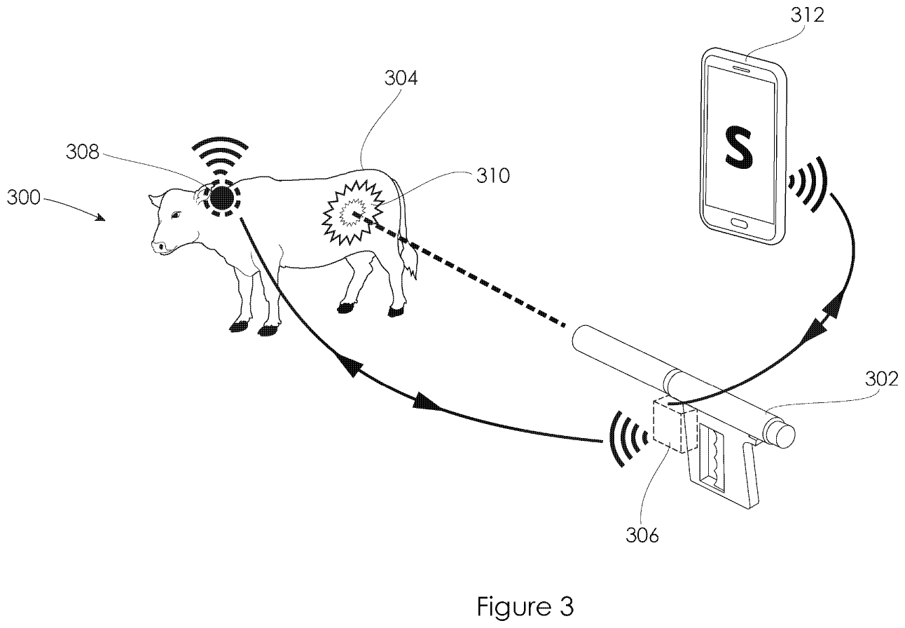

FIG. 3 is a schematic representation 300 of a treating apparatus 302 and an animal 304 wearing an electronic sensor 308 where the treating apparatus communicates with a remote smartphone 312. In this instance, the receiver 306 is arranged to receive data from the electronic sensor 308 and, in addition, transmit and receive data from a wireless enabled communication device 312, such a smartphone.

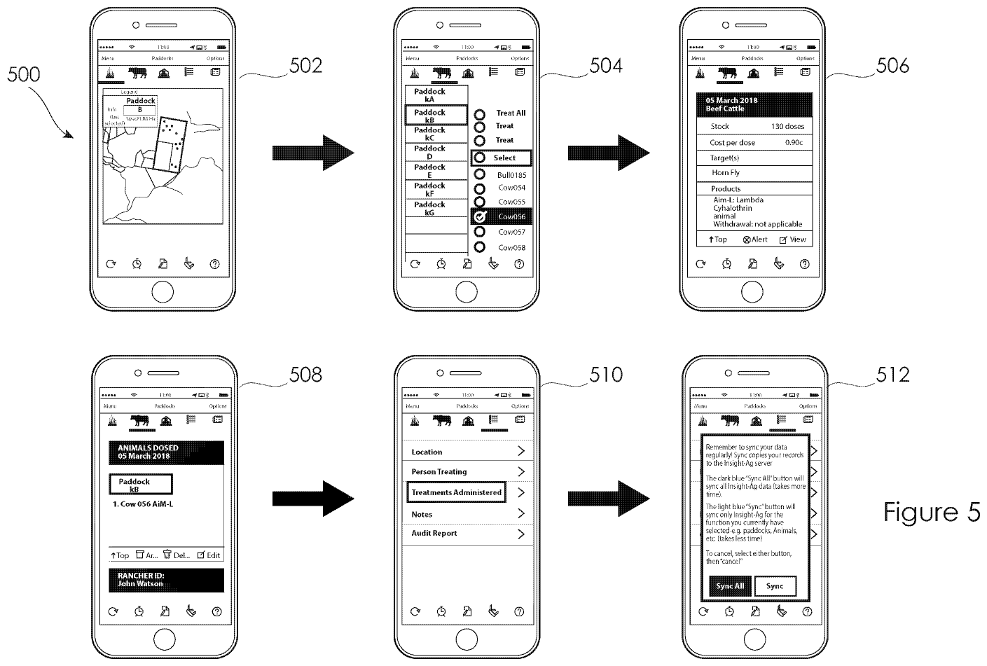

In this embodiment, records of treatment may be sent to device 312 for storage. A history of treatment and health related records is built using an application running on device 312. Furthermore, a user interface provided via the app on the device 312 can be used to select specific projectiles in the launcher to provide specific treatment. More details of the smartphone application are provided with reference to FIG. 5.

In alternative embodiments, the treating apparatus is installed on a vehicle that is capable to move across the farm's terrain and approach animals to deliver treatment. The vehicle may be human-controlled or autonomous. The vehicle may be a ground-based vehicle or an airborne based vehicle, such as a drone.

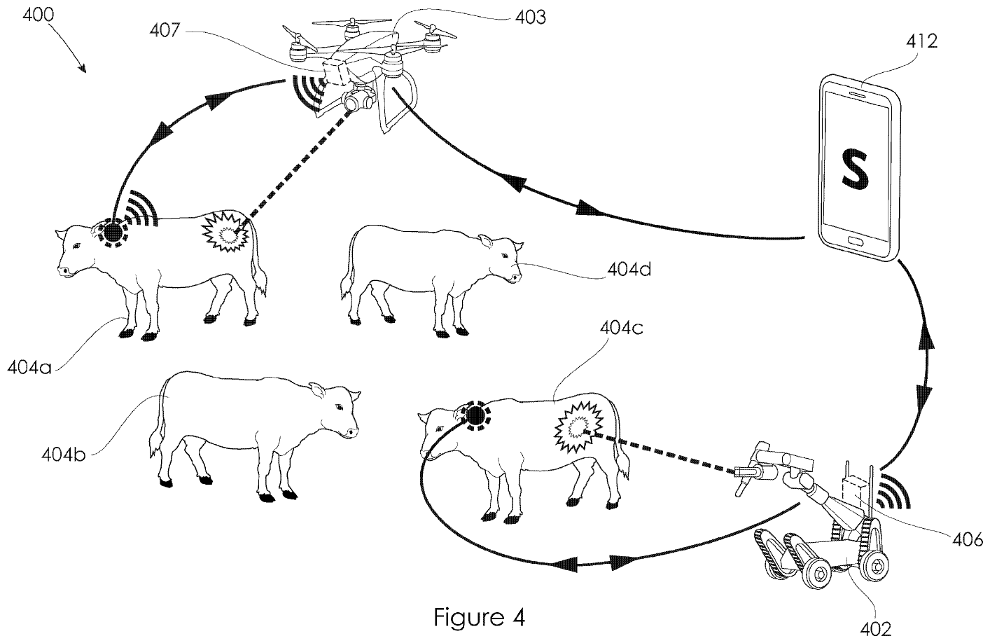

FIG. 4 shows a schematic representation 400 of a plurality of animals 404 in a farm environment and two treating apparatus 402, 403 with respective receiver modules 406, 407. A first treating apparatus 402 is mounted on a ground-based vehicle and a second treating apparatus 403 is mounted on a drone. The ground-based vehicle and the drone can operate autonomously and scan animals in the farm with the aim of actuating treatment through the treating apparatus on board. Data is exchanged between the electronic sensors on the animals and the electronic receiver modules 406, 407 so that the required treatment agents can be automatically selected. Treatment is administered automatically by the automated vehicle and a record of treatment is stored in the local memory of the receiver for future download/upload or sent to a remote communication device 412. In some instances, the vehicles can operate autonomously.

FIG. 5 shows a series of display screens of an application running on a smartphone used as part of the electronic receiver module. The smartphone application can provide a variety of functions to simplify locating animals on the farm, administering one or more treatments and maintaining treatment records for the one of more animals.

The ‘Paddock Map’ screen 502 allows the farmer to visualize a virtual map of the paddock/farm and locate animals that require treatment. The application may also be preloaded with data related to the animals present in a particular area on the farm and specific data of treatments associated with the animals.

The screens ‘Preselect Animals’ 504 and ‘Animal Treatments’ 506 can support the farmer with identifying animals that require treatment before heading out in the farm to administer the treatment and select a set of treatments for loading into the launcher amongst a range of treatments available at the farm. The ‘Summary’ 508 and ‘Reporting’ 510 screens allow to access recorded information about and history of treatment for a specific animal or a group of animals. In addition, the application provides guides to support farmers in delivering the correct treatment at the most suitable time. The application also provide sync capabilities with a distributed health monitoring platform 512.

In embodiments, the display of the smartphone and software application provide the user interface that communicates with the reader through its communication interface. This solution allows to keep the electronics in the reader simple and low cost and may be suitable, for example, to retrofit existing launchers that do not have communication capabilities. The software application smartphone also provides first level storage and processing of treatment records. In other embodiments, such as the one shown in FIG. 1, the user interface is provided on the launcher through a liquid crystal display permanently fixed on the device. It will be understood that these two solutions are not mutually exclusive and, even when a user interface is provided on the launcher, the electronic reader module still has communication capabilities to transmit and receive data from a user personal communication device, such as a smartphone or a smart watch.

Referring now to FIG. 6, there are shown a number of screenshots of the user interface provided on the screen of a smartphone. FIG. 5 shows a number of screens provided by a software application running on the smartphone. The paddock map screen allows identifying areas of the paddock where animals may be located. Once the area has been identified, the software application allows to preselect specific animals, or categories of animals, for treatment using a menu. An additional screen is provided by the application to allow pre-selection of the treatment to be provided to the selected animals. The application also provides, information screens that can summarise data on treatment provided during a day or any other specified period of time and produce treatment reports for one or more animals.

FIG. 6 shows the interface provided while treating animals in the farm. In this embodiment the smartphone is mounted to the launcher and an image of the animal being targeted is displayed on the smartphone screen. The smartphone may communicate with an external reader module to retrieve information on the current treatment state of the animal being targeted. Alternatively, the smartphone may host the electronic reader module internally and retrieve the information from the internal memory. The animal is identified by communication between the electronic reader module and the electronic sensor on the animal. The green reticule in FIG. 6(a) shows an animal that requires treatment. FIG. 6(b) shows the discharge of the launcher has been detected and a dosing of the targeted animal has been recorded. The user may then be asked to confirm or reject this record. In FIG. 6(c) the animal being targeted has already been treated with the selected treatment and the red reticle prompts the user not to dose the animal again.

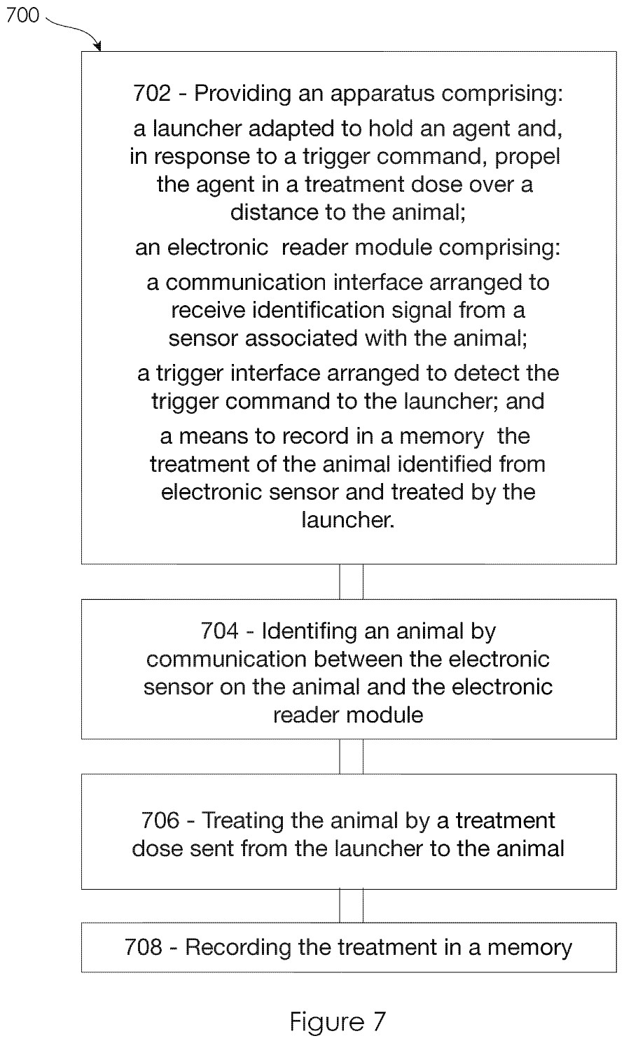

FIG. 7 is a flow diagram presenting a list of steps to perform a method of remotely treating an animal and recording treatment of the animal.

A treating apparatus in accordance with embodiments is provided (702). As the treating apparatus approaches the electronic sensor of one or more animals, the animal is identified (704), by communication between the electronic sensor on the animal and the communication interface on the treating apparatus . The animal is treated by a treatment dose sent from the launcher to the animal (706). Subsequently, a treatment is stored in the memory of the electronic reader module, or transmitted to the electronic sensor or the user's personal communication device for storage (708).

The electronic reader provides the user with the option of selecting an agent from a list of projectiles available in the launcher. Treatment history data may be made available on the display of the user interface and a specific projectile may be recommended based on the data. For example, different projectiles for different treatments can be loaded in the launcher. The type of projectiles may be related to the time of the year, season, or specific epidemics that are spreading amongst animals.

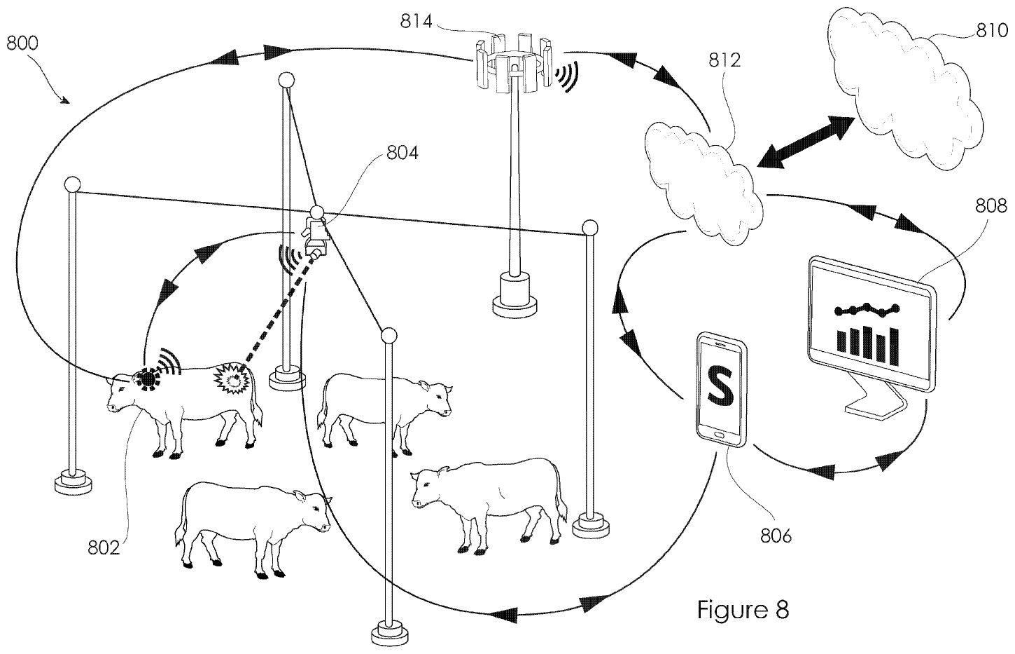

FIG. 8 is a schematic representation of a smart farm 800 that uses one or more apparatus for remotely treating an animal ad described above with reference to FIGS. 1 to 7. The system allows managing health conditions of a number of animals wearing electronic sensors 802 and treat the animals through an apparatus 804 for remotely treating the animals connected to a system pulleys and ropes connected. Treatment data is recorded by the by a receiver hosted in a mobile communication device 806 and subsequently transmitted to a server 808 that implements a health monitoring platform. The health monitoring platform can also be hosted in a cloud environment 810 that communicates to the server 808 via a communication network 812. The communication between the treatment agent, delivery apparatus 804, the electronic sensors and the electronic reader can be facilitated by a local communication tower dedicated to managing the smart farm.

### Launcher

The launcher may be handheld, anchored or position in stationary location, or associated with a mobile platform such as a vehicle, drone, or mounted on a pulley system that assists with positioning the launcher optimally for delivery of the dosage projectile.

The launcher can be an air or gas-powered launcher. The launcher can include a magazine or reservoir for accepting a plurality of projectiles. Administering an agent to an animal is accomplished by a person or user aiming the launcher containing one or more projectiles at the animal and launching a projectile at the animal with a velocity sufficient to deliver the agent to the animal. For non-skin piercing projectiles, this allows the contents of the projectile to be deposited onto the skin of the animal, allowing the agent to be located or absorbed through the skin of the animal.

The dosage projectiles may have sufficient volume to contain a unit dosage for treating the animal. The dosage is typically calculated to correspond to a certain minimum weight of animal to which a pesticide is to be administered. If larger animals are to be treated, the number of projectiles per animal may be increased accordingly. Alternatively, a single projectile dosage for all animal weights may be preserved by alteration of the formulation concentration of the active pharmaceutical agent.

The launcher can have a selector button which allows a user to pre-select the number of projectiles to be launched at the single pull of a trigger of the launcher, thereby allowing larger animals to be treated with the correct dose required, merely by selecting the number of projectiles to be launched substantially simultaneously. This has the advantage that the animal does not have a chance to escape following the first firing of the launcher, as the projectiles reach it substantially simultaneously. Launching one projectile at a time may result in the animal fleeing, making it difficult to track down the same animal and administer a second (or different) dose.

Similarly, it may be necessary to treat an animal with a combination of agents. This may be accomplished by using a projectile containing a combination of agents. However, it is not always possible to produce a projectile having two or more different agents therein, due to adverse reactions occurring between such agents when they are co-mixed.

The dosage projectiles can contain one or more biologically active agents for treating an animal.

The biologically active agent is typically present at a concentration (% v/v) of from about 0.1% to 20%. It will be appreciated that the concentration of the biologically active agent will be related to the dosage required for a particular size of animal.

The biologically active agent can be a pharmacological agent such as a veterinary pharmaceutical for treating insect pests or disease.

The biologically active agent can be a health supplement such as a vitamin or mineral.

The biologically active agent can be a vaccine or immunogenic compound. The biologically active agent may also include protein-based agents, such as crude or purified cell lysates, sub-unit vaccines, protein-based antigen display systems, antigens, peptides, oligopeptides, or polypeptides.

The biologically active agent may include drugs such as contraceptives, analgesics, anti-inflammatories, vasodilators, bronchodilators, diuretics, anti-histamines, tranquilizers, anti-fungals, vitamins, muscle relaxants, and anti-virals, anti-parasitic compositions, anthelmintics, acaricides, insecticides, and the like.

The biologically active agent may include a hormone such as a progesterone, estrogen, testosterone, derivatives thereof, and/or combinations of such hormones.

The biologically active agent may be poison or toxin used to kill an animal follow administration or consumption.

The treatment of the animal may be for a parasite, pest, illness, nutritional deficiency, vaccination, pain management, management of fever (pyrexia), bacterial or viral infections, growth promotion, hormones, steroids, fertility enhancing agents or contraceptive agents, sterilization, appetite stimulants, rumen activity modulators.

### Pests

The target pest may be an insect horn fly, face fly, stable fly, heel fly, warble fly, bot fly, house fly, horse fly, deer fly, blow fly, mosquito, midge, flea or louse.

The target pest may be an arachnid such as a tick or mite

The target pest may be a helminth such as a roundworm, stomach worm, tapeworm, or trematoda such as a flatworm or fluke.

The target pest may be a protozoan such as a coccidian.

In some embodiments the target pest may be an adult pest, a larvae, pupae, egg or any combination thereof.

### Dosage Projectiles

The dosage projectile may be a dosage dart, skin piercing dosage projectiles, non-skin piercing dosage projectile or a measured bolus of powder or gel.

Examples of dosage darts suitable for the present invention can be found in U.S. Pat. No. 7,795,263.

Non-skin piercing dosage projectiles marketed under the name VetCap® as described in WO 2008/052263 can be used with the launcher described herein. It should be noted that the dosage projectile does not require the use of a transdermal agent with the pesticides but a transdermal agent may optionally be employed for treating certain pests.

Suitable dosage projectiles are made from a substance such as, but not limited to, hydrophilic colloidal materials such as, gelatin, albumin, gum arabic, alginate, casein, agar or pectins, or combinations thereof. The projectile can also be made from a synthetic organic compound such as, but not limited to, plastics, synthetic polymers, polyesters, polylactic acid, starch copolymers, high molecular weight polyvinylalcohol, un-stabilized polyethelyne, un-stabilized polypropylene, biodegradable polymers, compostable polymers, biopolymers (including those comprised of polylactic acid), polystyrene, polypropylene, polyethylene, polycarbonate, polyamide, polysulfane, polyvinylchloride, resinous compounds such as (fibreglass or Perspex derivatives or combinations thereof, waxes and hardeners such as carnauba, candelilla, bees, paraffin, stearic acid, or combinations thereof.

The dosage projectile includes at least one agent such as a pesticide. The pesticide can be encapsulated in a controlled-release coating prior to inclusion in the dosage projectile thereby allowing the controlled release of the pesticide within an animal to be treated animal, once it has passed transdermally into the blood or lymphatic system of the animal. The controlled-release coating may be selected from controlled release compositions known in the field.

The viscosity of the projectile contents can be such that the contents do not run off the skin, fur or coat of the animal prematurely before treatment has occurred. Accordingly, the projectile may also include a thickening agent, such as a starch-like compound, inert polymer, gel, or an oil-based composition such as sesame seed oil, if required.

The pesticides contained in the projectile can be in different forms and/or concentrations, depending on the formulation, the carrying capacity, and solubility and release characteristics desired, for example as neutral molecules, components of molecular complexes, and pharmaceutically acceptable salts, free acids or bases, or quaternary, salts thereof. Simple derivatives of the pesticides mentioned herein, such as pharmaceutically acceptable ethers, esters, amides and the like which have desirable retention and release characteristics in vivo, and enzymes, pro-active forms, pro-drugs and the like, can also be employed as required.

The dosage projectiles may include additional components to enhance the effectiveness of the agents or to reduce discomfort to an animal.

In some embodiments the dosage projectile may include a transdermal carrier. The term ‘transdermal carrier’ or refers to any material known in the art as being suitable for transdermal pesticide administration and includes any polymeric material into which a pesticide may be solubilised in combination or admixture with the other ingredients to form a composition. The term also includes enhancers, solvents, co-solvents, carriers and other types of additives useful for facilitating transdermal pesticide delivery, or adhesives for ensuring adhesion of the contents of the projectile to the skin, coat or fur of a target animal.

The amount of pesticide to be complexed with the transdermal carrier will vary depending on the particular pesticide, and the time span for which the pesticide effective. Normally, the amount of pesticide in the transdermal system can vary from about 0.1% to about 50%, or even from about 0.1% to about 30% by weight based on the dry weight of the total carrier composition. Persons skilled in the field of the invention will be able to determine the adequate amounts required for each application, as required.

It is to be appreciated that the order of steps, the amounts of the ingredients, and the amount and time of mixing may be important process variables which will depend on the specific polymers, pesticides, solvents and/or co-solvents, enhancers, additives and/or excipients used in the composition.

The transdermal carrier, if used, is typically used in an amount of about 1% to about 95%, and preferably from about 10% to about 75%, by weight based on the weight of the total carrier composition.

The transdermal carrier composition can also contain one or more solvents and/or co-solvents known in the art.

Suitable solvents and co-solvents include volatile substances or compositions such as alcohols, aromatic hydrocarbons such as benzene derivatives, lower molecular weight alkanes and cycloalkanes, alkanoic acid esters, polyhydric alcohols, which include glycols, triols and polyols such as ethylene glycol, diethylene glycol, propylene glycol, dipropylene glycol, trimethylene glycol, butylene glycol, polyethylene glycol, hexylene glycol, polyoxethylene, glycerin, trimethylpropane, sorbitol, polyvinylpyrrolidone, glycol ethers such as ethylene glycol monoethyl ether, glycol esters, glycol ether esters such as ethylene glycol monoethyl ether acetate and ethylene glycol diacetate; saturated and unsaturated fatty acids, mineral oil, silicone fluid, lecithin, retinol derivatives and the like, and ethers, esters and alcohols of fatty acids, or combinations and mixtures thereof.

Although the exact amount of solvents and co-solvents that may be used in the carrier composition depends on the nature and amount of the other ingredients, such amount typically ranges from about 0.1% to about 50%, and preferably from about 0.1% to about 30% by weight, and more preferably from about 1% to about 20%, by weight based on the dry weight of the total carrier composition.

The transdermal carrier is typically selected so that it may be readily absorbable by the skin of an animal without causing undue itching, irritation, or toxic effects to the animal. Selection of the transdermal carrier will also depend on the pesticide to be delivered to an animal and also the type of animal to be treated, or the intended delivery site on an animal. Thus, the transdermal carrier composition may be selected to suit the charge, size, hydrophobicity, hydrophilicity, amphipathicity, pI, pH, decay rate, or other relevant criteria of the pesticide to be carried transdermally, while also being readily absorbable through the skin of an animal.

Typically, the transdermal carrier includes compounds such as isopropyl alcohol, dipropylene glycol methyl-ether, butylated hydroxytoluene dipropylene glycol monomethyl-ether, methylene chloride, 1-methoxy 2-propanol (glysolv PM/Icinol PM), Ethylene glycol monobutylether (butyl glyxolv/butyl icinol), Butyl di glysolv (butyl-icinol), Transcutol, propylene glycol (PG), N-methyl-2 pyrrolidone (NMP), diethyl ether, ethanol, acetonitrile, ethyl acetate, benzyl alcohol and a combination of natural oils. ethylene glycol, propylene glycol, dimethyl polysiloxane (DMPX), oleic acid, caprylic acid, 1-octanol, ethanol (denatured or anhydrous), liposomal compositions, suitable plant oils, such as Aloe vera derivatives or sesame seed oil or derivatives thereof, acrylic polymers, rubber-based polymers, polysiloxane-based polymers, polyvinylpyrrolidone-based polymers, dimethylsulfoxide (DMSO), dimethylformamide (DMF), lecithin, Transfersomes®, ethosomes, azone, castor oil derivatives, such as ethoxylated castor oil, jojoba oil derivatives, corn oil derivatives, emu oil derivatives, or other suitable carriers.

An enhancer can be incorporated into the carrier composition. The term ‘enhancers’ as used herein refers to substances used to increase permeability and/or accelerate the delivery of an active agent through the skin of an animal, and include monohydric alcohols such as ethyl, isopropyl, butyl and benzyl alcohols; or dihydric alcohols such as ethylene glycol, diethylene glycol, or propylene glycol dipropylene glycol and trimethylene glycol; or polyhydric alcohols such as glycerin, sorbitol and polyethylene glycol, which enhance drug solubility; polyethylene glycol ethers of aliphatic alcohols (such as cetyl, lauryl, oleyl and stearly) including polyoxyethylene-4-lauryl ether, polyoxyethylene-2-oleyl ether and polyoxyethylene-10-oleyl ether; vegetable, animal and fish fats and oils such as cotton seed, corn, safflower, olive and castor oils, squalene, and lanolin; fatty acid esters such as propyl oleate, decyl oleate, isopropyl palmitate, glycol palmitate, glycol laurate, dodecyl myristate, isopropyl myristate and glycol stearate which enhance drug diffusibility; fatty acid alcohols such as oleyl alcohol and its derivatives; fatty acid amides such as oleamide and its derivatives; urea and urea derivatives such as allantoin which affect the ability of keratin to retain moisture; polar solvents such as dimethyldecylphosphoxide, methyloctylsulfoxide, dimethyllaurylamide, dodecyl pyrrolidone, isosorbitol, dimethylacetonide, dimethylsulfoxide, decylmethylsulfoxide and dimethylformamide; salicylic acid; benzyl nicotinate; or higher molecular weight aliphatic surfactants such as lauryl sulfate salts, esters of sorbitol and sorbitol anhydride such as polysorbate. Other suitable enhancers include oleic and linoleic acids, triacetin, ascorbic acid, panthenol, butylated hydroxytoluene, tocopherol, tocopherol acetate, tocopheryl linoleate.

If enhancers are incorporated into the carrier composition, the amount typically ranges up to about 35%, and preferably from about 0.05% to about 20%, by weight based on the dry weight of the total carrier composition.

### Pesticides

It is contemplated that any pesticide which is deliverable to an animal to produce a desired, usually beneficial, effect may be used in the methods and treatment regimes of the present invention. It should be noted that the pesticides can be used singularly or in combinations or mixtures as required.

Examples of pesticides include parasiticides and/or anthelmintics which include, but are not limited to the following:

Macrocyclic lactones including the avermectins and milbemycins, for example Ivermectin, eprinomectin, moxidectin, selamectin, doramectin, milbemycin, abamectin, cydectin and emamectin benzoate.

Synthetic pyrethroids such as flumethrin, deltamethrin, cypermethrin, cyfluthrin, lambda cyhalothrin, fenvalerate, alphacypermethrin and pyrethrin.

Insect growth regulators such as pyriproxifen, methoprene, cyromazine, lufenuron, diflubenzuron, fluazuron, dicyclanil and fluazuron.

Anthelminitics such as fipronil, imidacloprid, rotenone, magnesium flurosilicate, piperonyl butoxide, spinosyns and other suitable benzimidazole anthelmintics and immunomodulators (e.g. Levamisole).

Anthelmintic, anti-trematodal, anticestodal, or anti-parasitic/parasiticidal agents such as albendazole, levamisole, mebendazole, pyrantel, praziquantel, moxidectin, ivermectin, oxamniquine, metrifonate, piperazine, thiabendazole, tiabendazole, diethylcarbamazine, pyrantel, niclosamide, doramectin, eprinomectin, morantel, oxfendazole, dichlorvos, chlorsulon and selamectin.

The pesticide may be a pro-insecticide being a compound that is metabolized into an active insecticide after entering the host or target insect. The pro-insecticide may be derived from a microbially produced compounds for example halogenated pyrroles, an example of this class being chlorfenapyr, caricides, nikkomycins, thuringiensin, macrocyclic lactones, acaricides, tetranactin, avermectin, acaricides, abamectin, doramectin, eprinomectin, ivermectin, selamectin, milbemycin, acaricides, milbemectin, milbemycin oxime, moxidectin, bridged diphenyl acaricides, azobenzene, benzoximate, benzyl benzoate, bromopropylate, chlorbenside, chlorfenethol, chlorfenson, chlorfensulphide, chlorobenzilate, chloropropylate, cyflumetofen, dicofol, diphenyl sulfone, dofenapyn, fenson, fentrifanil, fluorbenside, proclonol, tetradifon, tetrasul, carbamate acaricides, benomyl, carbanolate, carbaryl, carbofuran, methiocarb, metolcarb, promacyl, propoxur, oxime carbamate caricides, aldicarb, butocarboxim, oxamyl, thiocarboxime, thiofanox, dinitrophenol acaricides, binapacryl, dinex, dinobuton, dinocap, dinocap-6, dinocton, dinopenton, dinosulfon, dinoterbon, DNOC, formamidine acaricides, amidines, amitraz, chlordimeform, chloromebuform, formetanate, formparanate, mite growth regulators, clofentezine, diflovidazin, dofenapyn, fluazuron, flubenzimine, flucycloxuron, flufenoxuron, hexythiazox, organochlorine acaricides, bromocyclen, camphechlor, dienochlor, endosulfan, lindane, organophosphorus acaricides, organophosphate acaricides, chlorfenvinphos, crotoxyphos, dichlorvos, heptenophos, mevinphos, monocrotophos, TEPP, tetrachlorvinphos, organothiophosphate acaricides, amidithion, amiton, azinphos-ethyl, azinphos-methyl, azothoate, benoxafos, bromophos, bromophos-ethyl, carbophenothion, chlorpyrifos, chlorthiophos, coumaphos, cyanthoate, demeton, demeton-O, demeton-S, demeton-methyl, demeton-O-methyl, demeton-S-methyl, demeton-S-methylsulphon, dialifos, diazinon, dimethoate, dioxathion, disulfoton, endothion, ethion, ethoate-methyl, formothion, malathion, mecarbam, methacrifos, omethoate, oxydeprofos, oxydisulfoton, parathion, phenkapton, phorate, phosalone, phosmet, phoxim, pirimiphos-methyl, prothidathion, prothoate, pyrimitate, quinalphos, quintiofos, sophamide, sulfotep, thiometon, triazophos, trifenofos, vamidothion, phosphonate acaricides, trichlorfon, phosphoramidothioate acaricides, isocarbophos, methamidophos, propetamphos, phosphorodiamide caricides, dimefox, mipafox, schradan, organotin acaricides, azocyclotin, cyhexatin, fenbutatin, phenylsulfamide acaricides, dichlofluanid, phthalimide acaricides, dialifos, phosmet, pyrazole acaricides, acetoprole, fipronil, tebufenpyrad, vaniliprole, pyrethroid acaricides, pyrethroid ester caricides, acrinathrin, bifenthrin, cyhalothrin, cypermethrin, alpha-ypermethrin, fenpropathrin, fenvalerate, flucythrinate, flumethrin, fluvalinate, tau-fluvalinate, permethrin, pyrethroid ether acaricides, halfenprox, pyrimidinamine acaricides, pyrimidifen, pyrrole acaricides, chlorfenapyr, quinoxaline acaricides, chinomethionat, thioquinox, sulfite ester caricides, propargite, tetrazine acaricides, clofentezine, diflovidazin, tetronic acid acaricides, spirodiclofen, thiocarbamate acaricides, fenothiocarb, thiourea acaricides, chloromethiuron, diafenthiuron, unclassified acaricides, acequinocyl, amidoflumet, arsenous oxide, bifenazate, closantel, crotamiton, disulfiram, etoxazole, fenazaflor, fenazaquin, fenpyroximate, fluacrypyrim, fluenetil, mesulfen, MNAF, nifluridide, pyridaben, sulfiram, sulfluramid, sulfur triarathene.

Cholinergic agents such as acetylcholine, arecoline, bethanechol, carbachol, choline, methacoline, muscarine and pilocarpine.

Anti-cholinergic agents such as atropine, eucatropine and procyclidine.

The amounts of the pesticide to be used in each dosage projectile may be determined by methods known to persons skilled in the art. Amounts typically range from about 0.05 mg to about 20,000 mg, and preferably from about 0.1 mg to about 1,000 mg, depending on the pesticide, the target, the animal species, the size of the animal. In certain embodiments of the invention, the pesticides may be included in a range from about 0.1 to about 500 mg per mammal per 50 kg body weight.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An apparatus for remotely treating an animal with an agent, the apparatus comprising:
a launcher adapted to hold an agent and, in response to a trigger command, propel the agent in a treatment dose over a distance to the animal;
an electronic reader module comprising:
a communication interface arranged to receive an identification signal from a sensor associated with the animal;
a trigger interface arranged to detect the trigger command to the launcher; and
a means to record in a memory the treatment of the animal identified from electronic sensor and treated by the launcher.

2. The apparatus according to claim 1, wherein the launcher comprises:
a chamber adapted to receive the agent;
a launcher barrel or nozzle for facilitating launching of a treatment dose of the agent; and
a propulsion assembly for propelling the treatment dose through the barrel or nozzle to an animal in response to the trigger command.

3. The apparatus of claim 1 wherein the treatment dose is contained in a dosage projectile, a liquid, a spray, a dart, a gel or a bolus.

4. The apparatus of claim 1, wherein, when a treatment dose is launched by the launcher, a signal is detected by the electronic reader module and the electronic reader module initiates recording of the record of treatment of the animal in the memory.

5. The apparatus of claim 1, wherein the memory is hosted on the reader module.

6. The apparatus of claim 1, wherein the means to record the treatment comprise a transmitter to transmit treatment data for recording.

7. The apparatus of claim 6, wherein the memory is embedded into the electronic sensor and the communication interface is arranged to transmit a record of treatment to the electronic sensor for storage in the memory.

8. The apparatus of claim 1, wherein the electronic reader module is integrated with the launcher or removably mounted to the launcher.

9. The apparatus of claim 1, wherein the electronic reader module comprises two devices, a first device integrated with the launcher and a second device removably mounted onto the launcher.

10. The apparatus of claim 1, wherein the electronic reader module comprises a personal communication device removably mounted to the launcher by means of a mounting bracket or a fastener.

11. The apparatus of claim 10, wherein the personal communication device interacts with a detector located on the launcher, or the communication interface is hosted in the personal communication device and the detector is arranged to initiate communication between the electronic sensor on the animal and the communication interface.

12-13. (canceled)

14. The apparatus of claim 1, wherein the detector comprises a proximity sensor which, when in proximity of the animal, initiates communication between the communication interface and the sensor, and/or the detector comprises a light or noise frequency emitting source that is detectable by the sensor on the animal and triggers the initiation of communication between the communication interface and the sensor.

15. (canceled)

16. The apparatus of claim 14 wherein the light emitting source comprises a laser light emitting device.

17. The apparatus of claim 1, wherein the identification signal received by the launcher encodes an identification code of the animal.

18. The apparatus of claim 17, wherein the signal comprises a light signal emitted by a light source that can be read by a visual system on-board of the detector, or wherein the signal comprises data associated with the animal, such as identification data, treatment history, real time physiological data, or health-related historical data, or wherein the electronic sensor is activated when the detector is located within a predetermined distance and starts emitting a signal encoding identification information for the animal.

19-20. (canceled)

21. The apparatus of claim 1, further comprising a user interface arranged to enable user selection of a specific agent from a list of available agents that can be loaded into the launcher.

22. The apparatus of claim 21, wherein the user interface is arranged to prompt the user with a history of treatment for the animal, or wherein the user interface comprises a user display arranged to show the history of treatment for the identified animal and provide a selection menu of agents that can be loaded into the launcher, or wherein the user interface is provided on a display of a user communication device and data related to treatment of the animal are exchanged between the communication interface of the launcher and the user communication device.

23-24. (canceled)

25. The apparatus of claim 1, wherein the apparatus is arranged to be installed on a vehicle that is capable to move or fly across a terrain of a farm terrain and approach animals to deliver the treatment dose, or wherein the apparatus is handheld, anchored or positioned in stationary location, or associated with a mobile platform comprising a vehicle, drone or cable and pulley system used to orientate the apparatus for delivery of the treatment dose.

26. (canceled)

27. A method of remotely treating an animal, the method comprising:
providing an apparatus according to claim 1;
identifying an animal by communication between the sensor on the animal and the electronic reader module;
treating the animal by a treatment dose sent from the launcher to the animal; and
recording the treatment in a memory.

28. The method of claim 27, wherein recording the treatment comprises the step of transmitting the record of treatment to a remote server or a user communication device and storing the record of treatment in a memory of the remote server or user communication device.

29-31. (canceled)
